(19) United States
(12) Patent Application Publication
(10) Pub. No.: US 2024/0306978 A1
(43) Pub. Date: Sep. 19, 2024

(54) CURRENT SOURCE ESTIMATED TIME DETERMINATION METHOD, INFORMATION PROCESSING APPARATUS, AND COMPUTER-READABLE MEDIUM

(71) Applicants: Miyako ASAI, Osaka (JP); Nobukazu NAKASATO, Miyagi (JP); Akitake KANNO, Miyagi (JP)

(72) Inventors: Miyako ASAI, Osaka (JP); Nobukazu NAKASATO, Miyagi (JP); Akitake KANNO, Miyagi (JP)

(73) Assignees: Ricoh Company, Ltd., Tokyo (JP); TOHOKU UNIVERSITY, Sendai-shi (JP)

(21) Appl. No.: 18/598,434

(22) Filed: Mar. 7, 2024

(30) Foreign Application Priority Data
Mar. 17, 2023 (JP) 2023-043620

(51) Int. Cl. A61B 5/00; A61B 5/245; A61B 5/372

(52) U.S. Cl. CPC A61B 5/4094; A61B 5/245; A61B 5/372

(57) ABSTRACT

A current source estimation time determination method includes: determining an analysis interval in which a characteristic waveform is obtained with respect to one or more pieces of waveform data acquired by a sensor; estimating a current source with respect to a signal in the analysis interval determined at the determining; detecting a continuous movement interval in which the current source continuously moves in the analysis interval, using coordinates of the current source estimated at the estimating; and determining a single current source estimation time from the continuous movement interval detected at the detecting.

### CROSS-REFERENCE TO RELATED APPLICATIONS

The present application claims priority under 35 U.S.C. § 119 to Japanese Patent Application No. 2023-043620, filed on Mar. 17, 2023. The contents of which are incorporated herein by reference in their entirety.

### BACKGROUND OF THE INVENTION

### 1. Field of the Invention

The present invention relates to a current source estimation time determination method, an information processing apparatus, and a computer-readable medium.

### 2. Description of the Related Art

Conventionally, it is extremely important to analyze a characteristic waveform in analysis of magnetoencephalography or electroencephalography. However, with the advancement of technologies, sampling frequencies and the number of sensors at the time of measurement tends to increase, and therefore, a time needed to visually search for a waveform tends to increase.

For example, in diagnosis of epilepsy using magnetoencephalography in clinical practice, locality of an epilepsy lesion site is evaluated by using a method called an equivalent current dipole method. In the equivalent current dipole method, a current source (dipole) that generates a magnetic field that is measured on a scalp is estimated. To estimate the dipole, there is a need to narrow down a time (onset site) at which characteristic waveform information (Interictal Epileptiform Discharge (IED)) occurs from a sequence of times of a plurality of sensors and a sensor in which the waveform information appears.

In the present circumstances, a doctor manually searches for the IED and determines an onset site; however, a volume of magnetoencephalography data is enormous, and therefore, it is difficult to manually and accurately extract a sensor for individual IED and a time of the IED.

Further, when a doctor performs analysis, it is ideal to analyze the onset site of the IED by the equivalent current dipole method; however, it is known that a signal-to-noise ratio (S/N) of the onset site is low. Therefore, when the doctor performs analysis, in reality, the time of the IED is determined in a range from the onset site to a peak of the IED.

However, a current source estimation time determination method is not strictly defined even in a guideline provided by the academic society (Isao Hashimoto, Ryusuke Kakigi, Hideaki Shiraishi, Nobukazu Nakasato, Takashi Nagamine, Yutaka Watanabe: Guidelines for Clinical Applications of Magnetoencephalography, Japanese journal of clinical neurophysiology, 2005, 33-2, pp 69-86) or a guideline (Bagic A I, Knowlton R C, Rose D F, Ebersole J S, et al: American Clinical Magnetoencephalography Society Clinical Practice Guideline 1: recording and analysis of spontaneous cerebral activity, J Clin Neurophysiol 2011, 28, 1), and therefore, a doctor manually determines the time by individual judgement, which leads to variation.

To cope with this, Japanese Unexamined Patent Application Publication No. 2021-069929 discloses a system that, to more accurately determine a time at which characteristic waveform information (IED) appears and extract a sensor, detects a time of the IED and selects a sensor that are needed for the equivalent current dipole method by using a probability map of the IED.

However, according to the technology disclosed in Japanese Unexamined Patent Application Publication No. 2021-069929, there is a problem in that a current source estimation time determination method does not always conform to an evaluation criteria adopted by a doctor.

### SUMMARY OF THE INVENTION

A current source estimation time determination method includes: determining an analysis interval in which a characteristic waveform is obtained with respect to one or more pieces of waveform data acquired by a sensor; estimating a current source with respect to a signal in the analysis interval determined at the determining; detecting a continuous movement interval in which the current source continuously moves in the analysis interval, using coordinates of the current source estimated at the estimating; and determining a single current source estimation time from the continuous movement interval detected at the detecting.

### BRIEF DESCRIPTION OF THE DRAWINGS

FIG. 1 is a schematic diagram illustrating a configuration of a biological signal measurement system according to a first embodiment;

FIG. 2 is a block diagram schematically illustrating a functional configuration of a server;

FIG. 3 is a diagram illustrating an example of a hardware configuration of an information processing apparatus;

FIG. 4 is a diagram illustrating an example of a functional block configuration of the information processing apparatus;

FIG. 5 is a flowchart illustrating the flow of an estimation time detection process;

FIG. 6 is a flowchart illustrating the flow of a training process in a case where an IED probability map is generated by using machine learning;

FIG. 7 is a diagram illustrating an example of the IED probability map;

FIG. 8 is a diagram illustrating an example of the IED probability map that is subjected to a threshold process;

FIGS. 9A to 9C are schematic diagrams illustrating an example of a method of augmenting the number of sensors;

FIG. 10 is a flowchart illustrating the flow of the estimation time detection process;

FIG. 11 is a diagram illustrating an example of a trajectory of a dipole in an analysis interval;

FIG. 12 is a diagram illustrating an example of display in which the trajectory of the dipole in the analysis interval is superimposed on a brain slice image; and

FIG. 13 is a diagram illustrating an example of a threshold setting input user interface (UI) according to a second embodiment.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to determine a time of characteristic waveform information (IED) so as to conform to an analysis method adopted by a doctor.

Embodiments of a current source estimation time determination method, an information processing apparatus, and a non-transitory computer readable storage medium will be described in detail below with reference to the accompanying drawings. Further, the present invention is not limited by the embodiments below, and components in the embodiments below include one that can easily be thought of by a person skilled in the art, one that is practically identical, and one that is within an equivalent range. Furthermore, within the scope not departing from the gist of the following embodiments, various omission, replacement, and modifications of the components may be made.

The present embodiment has a feature as described below in terms of determination of a current source estimation time. Specifically, an interval that includes a characteristic waveform is detected, and an interval in which a current source (dipole) continuously moves in the detected interval is retrieved. With this configuration, it is possible to retrieve an interval in which an activity of the characteristic waveform is captured, and, by detecting a dipole near an onset in the interval, it is possible to automatically detect a time of an activity onset site with respect to the activity of the characteristic waveform.

When a doctor performs analysis, it is ideal to analyze an onset site of an IED by the equivalent current dipole method; however, it is known that an S/N of the onset site is low and, in reality, a time of the IED is determined in a range from the onset site to a peak of the IED. In the present embodiment, analysis is performed without limiting the analysis to the onset site.

### First Embodiment

### Overview of Biological Signal Measurement System

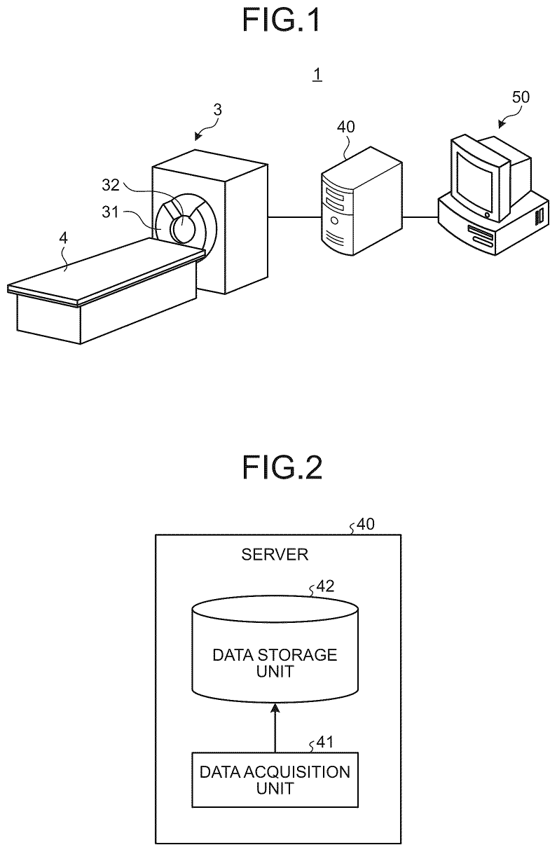

FIG. 1 is a schematic diagram illustrating a configuration of a biological signal measurement system according to a first embodiment. With reference to FIG. 1, an overview of a biological signal measurement system 1 according to the present embodiment will be described.

The biological signal measurement system 1 is a system that measures a plurality of kinds of biological signals (for example, a Magneto-encephalography (MEG) signal and an Electro-encephalography (EEG) signal) on a subject from a specific emission source (living body site), and displays the biological signals. Meanwhile, in the present invention, the biological signals to be measured are not limited to the magneto-encephalography signal and the electro-encephalography signal, but may be, for example, an electrical signal that is generated in response to heart activities (electrical signal that can be represented by an electro-cardiogram) or the like.

As illustrated in FIG. 1, the biological signal measurement system 1 includes a measurement apparatus 3 that measures one or more biological signals on a subject, a server 40 that records one or more kinds of biological signals that are measured by the measurement apparatus 3, and an information processing apparatus 50 that is a biological signal display apparatus that analyzes one or more kinds of biological signals that are recorded in the server 40. The measurement apparatus 3 is, for example, a magnetoencephalography measurement apparatus that collects measurement values of a brain's magnetic field, a timing at which a stimulus is applied, or the like. Meanwhile, in FIG. 1, the server 40 and an information processing apparatus 50 are separately illustrated, but, for example, it is possible to adopt a configuration in which at least a part of functions included in the server 40 is incorporated in the information processing apparatus 50.

In the example illustrated in FIG. 1, a subject (to-be-measured person) lies down on a measurement table 4 with face up while electrodes (or sensors) for electroencephalography are mounted on his/her head, and a head portion is inserted in a hollow 32 of a dewar 31 of the measurement apparatus 3. The dewar 31 is a holding container in an extremely low temperature environment using liquid helium, and a large number of magnetic sensors for magnetoencephalography are arranged inside the hollow 32 of the dewar 31. The measurement apparatus 3 collects electro-encephalography signals from the electrodes and magneto-encephalography signals from the magnetic sensors, and outputs data (hereinafter, may be referred to as “measurement data”) that includes the collected electro-encephalography signals and the collected magneto-encephalography signals to the server 40. The measurement data that is output to the server 40 is read, displayed, and analyzed by the information processing apparatus 50. In general, the dewar 31 in which the magnetic sensors are incorporated and the measurement table 4 are arranged in a magnetic shielding room, but illustration of the magnetic shielding room is omitted in FIG. 1 for the sake of convenience.

The information processing apparatus 50 is an apparatus that displays waveform data of the magneto-encephalography signals obtained from the plurality of magnetic sensors and waveform data of the electro-encephalography signals obtained from the plurality of electrodes in a synchronous manner on the same time axis. The electro-encephalography signal is a signal that represents electrical activity of a nerve cell (ion charge flow that occurs in dendrites of a neuron at the time of synaptic transmission) as a voltage value between the electrodes. The magneto-encephalography signal is a signal that represents minute magnetic field variation that occurs due to electrical activity of a brain. The brain's magnetic field is detected by a high-sensitive superconducting quantum interference device (SQUID) sensor. The electro-encephalography signal and the magneto-encephalography signal are examples of a “biological signal”.

FIG. 2 is a block diagram schematically illustrating a functional configuration of the server 40. As illustrated in FIG. 2, the server 40 includes a data acquisition unit 41 and a data storage unit 42.

The data acquisition unit 41 periodically acquires measurement data from the measurement apparatus 3. Here, the measurement data is each piece of waveform data that is measured by each of the magnetic sensors of the dewar 31 of the measurement apparatus 3.

The data storage unit 42 stores therein the measurement data that is acquired from the measurement apparatus 3.

### Hardware Configuration of Information Processing Apparatus

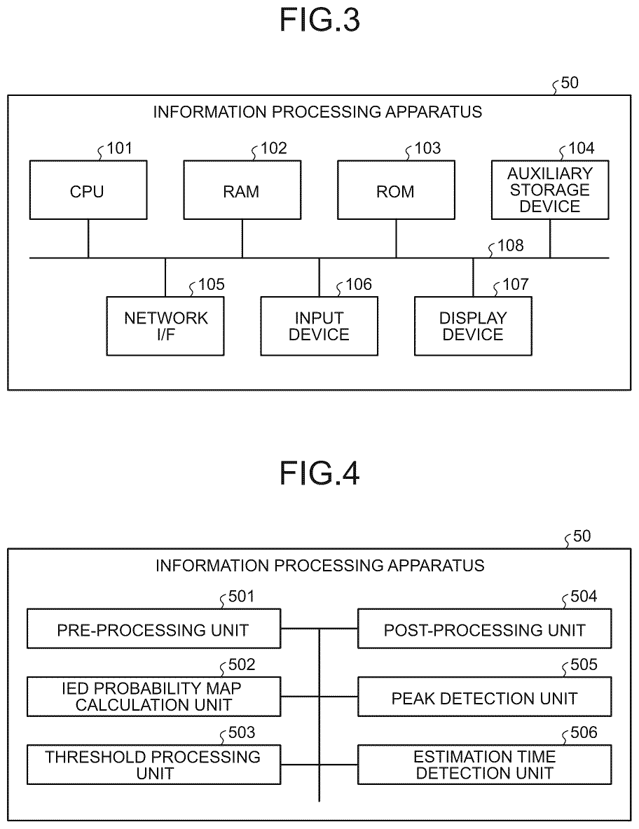

FIG. 3 is a diagram illustrating an example of a hardware configuration of the information processing apparatus 50. The hardware configuration of the information processing apparatus 50 will be described below with reference to FIG. 3.

As illustrated in FIG. 3, the information processing apparatus 50 includes a central processing unit (CPU) 101, a random access memory (RAM) 102, a read only memory (ROM) 103, an auxiliary storage device 104, a network interface (I/F) 105, an input device 106, and a display device 107, all of which are connected to one another via a bus 108.

The CPU 101 is an arithmetic device that controls entire operation of the information processing apparatus 50 and performs various kinds of information processing. The CPU 101 executes an information display program that is stored in the ROM 103 or the auxiliary storage device 104 and controls operation of displaying a measurement collection screen and an analysis screen.

The RAM 102 is a volatile storage device that is used as a work area of the CPU 101 and that stores therein main control parameters and information. The ROM 103 is a non-volatile storage device that stores therein a basic input-output program or the like. For example, it may be possible to store the information display as described above in the ROM 103.

The auxiliary storage device 104 is a storage device, such as a hard disk drive (HDD) or a solid state drive (SD). The auxiliary storage device 104 stores therein, for example, a control program for controlling operation of the information processing apparatus 50, various kinds of data and files that are needed for the operation of the information processing apparatus 50, and the like.

The network I/F 105 is a communication interface for performing communication with an apparatus, such as the server 40, on a network. The network I/F 105 is implemented by, for example, a network interface card (NIC) or the like that is compliant with transmission control protocol/Internet protocol (TCP/IP).

The input device 106 is an input function of a touch panel, a keyboard, a mouse, a user interface, such as an operation button, or the like. The display device 107 is a display device that displays various kinds of information. The display device 107 is implemented by, for example, a display function of a touch panel, a liquid crystal display (LCD), an organic electro-luminescence (EL), or the like. The display device 107 displays the measurement collection screen and the analysis screen, and updates the screen in accordance with input-output operation that is performed via the input device 106.

Meanwhile, the hardware configuration of the information processing apparatus 50 illustrated in FIG. 3 is one example, and it may be possible to add a different device. Further, the information processing apparatus 50 illustrated in FIG. 3 has the hardware configuration based on the assumption that the information processing apparatus 50 is a personal computer (PC) for example, but embodiments are not limited to this example, and a mobile terminal, such as a tablet, may be adopted. In this case, it is sufficient that the network I/F 105 is a communication interface with a wireless communication function.

### Functional Block Configuration and Operation of Information Processing Apparatus

FIG. 4 is a diagram illustrating an example of a functional block configuration of the information processing apparatus 50. The functional block configuration of the information processing apparatus 50 will be described below with reference to FIG. 4.

As illustrated in FIG. 4, the information processing apparatus 50 includes a pre-processing unit 501, an IED probability map calculation unit 502, a threshold processing unit 503, a post-processing unit 504, a peak detection unit 505, and an estimation time detection unit 506.

The pre-processing unit 501 performs pre-processing, such as extraction and augmentation of a sensor, down-sampling, application of a frequency filter, elimination of artifacts, a defective channel process, extraction of a time window, and standardization of magnetic field data.

As for the extraction and augmentation of a sensor, when an IED probability map (to be described later) is to be calculated, it may be possible to perform calculation by using only a group of a fixed number of sensors that are grouped in advance, in addition to a calculation method using all of sensors. As for grouping of sensors, it may be possible to form a group in accordance with an anatomical criterion, such as a temporal lobe or a frontal lobe, or it may be possible to simply form a group of an arbitrary number of sensors that are located close to each other. Furthermore, in a training process (to be described later), if the number of sensors at the time of calculation is smaller than the number of used sensors, it may be possible to expand fictional sensors.

The down-sampling is applied so as to adjust to a sampling frequency that is used at the time of training. The same applies to a frequency filter, and the same frequency filter that is applied to filtering at the time of training is to be applied. The filter that is frequently used is a low-pass filter at 35 Hz, a band-pass filter at 3 Hz to 35 Hz, or the like.

As for the elimination of artifacts, to eliminate cardiac artifacts or artifacts due to blink or body motion, ICA (see E. Javier, H. Roberto, A. Daniel, F. Alberto, and L. C. Miguel, “Artifact removal in magnetoencephalogram background activity with independent component analysis”, IEEE Trans Biomed Eng, vol. 54, no. 11, pp. 1965-1973, 2007), DSP (see K. Sekihara, Y. Kawabata, S. Ushio, S. Sumiya, S. Kawabata, Y. Adachi, and S. S. Nagarajan, “Dual signal subspace projection (DSP): a novel algorithm for removing large interference in biomagnetic measurements”, Journal of Neural Engineering, vol. 13, no. 3, p. 036007, 2016), or the like may be applied.

The defective channel process indicates a process of eliminating a sensor for which a magnetic field change that exceeds a threshold that is set in advance is observed, or a process for performing interpolation using peripheral sensor values.

As for the extraction of a time window, it may be possible to adopt a method of shift by a length corresponding to the time window without overlap, a method of overlapping a half of the length corresponding to the time window, a method of overlapping one-fourth of the length corresponding to the time window, or the like. In the case of overlap, an additive average is performed for the overlapping portion at the time of calculation of the IED probability map (to be described later).

As for the standardization of magnetic field data, standardization is applied such that an average in the extracted time window is 0 and dispersion is 1. It may be possible to use a normalization method such that a range of the magnetic field set in advance is −1 to 1, in addition to the standardization.

The IED probability map calculation unit 502 calculates a probability map of the characteristic waveform (Interictal Epileptiform Discharge (IED)).

The IED indicates a waveform, such as a Spike Wave, a Spike and Wave, a Poly Spiked Wave, or a Sharp Wave, which is characteristic of an epilepsy lesion site. In the present embodiment, the IED probability map calculation unit 502 calculates the probability map by using a machine trained model that is trained in advance. A method of generating the machine trained model will be described later.

The threshold processing unit 503 narrows down, from the IED probability map that is obtained by the IED probability map calculation unit 502, a region of a time and sensors in which IED probabilities are high, by using a threshold.

The post-processing unit 504 performs post-processing for extracting a sampling time pint and a sensor used for dipole estimation from the IED probability map that is subjected to a threshold process by the threshold processing unit 503. Further, if a plurality peaks are present in a peak detection method or a map (a case in which two or more lesion sites are present), the post-processing unit 504 performs post-processing for separating the map.

The peak detection unit 505 detects a peak by using the sampling point that is obtained in the process that is performed prior to the process performed by the peak detection unit 505.

Lastly, the estimation time detection unit 506 detects a current source estimation time by using a peak time that is obtained by the peak detection unit 505.

A flow of an estimation time detection process will be described below.

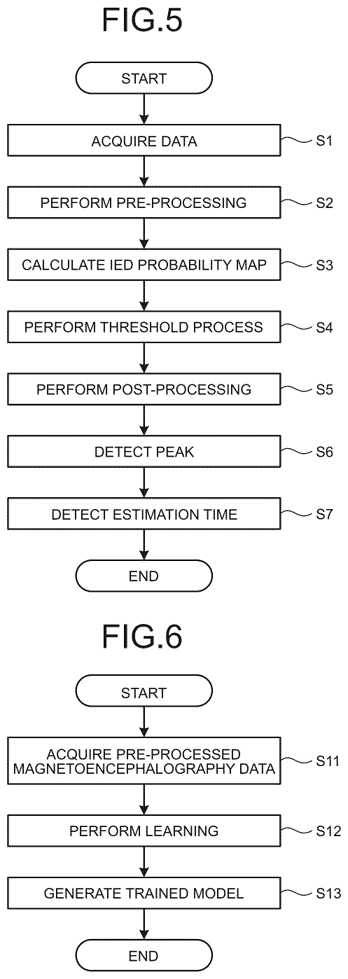

FIG. 5 is a flowchart illustrating the flow of the estimation time detection process. Before the process, the measurement apparatus 3 performs magnetoencephalography measurement, and outputs individual waveform data that is measured by each of the magnetic sensors of the dewar 31 to the server 40. The server 40 stores the individual waveform data that is measured by each of the magnetic sensors of the dewar 31 of the measurement apparatus 3 in the data storage unit 42.

As illustrated in FIG. 5, first, the pre-processing unit 501 acquires the individual waveform data that is measured by each of the magnetic sensors of the dewar 31 of the measurement apparatus 3 from the data storage unit 42 of the server 40 (Step S1).

Subsequently, the pre-processing unit 501 performs pre-processing, such as extraction and augmentation of a sensor, down-sampling, application of a frequency filter, elimination of artifacts, a defective channel process, extraction of a time window, and standardization of magnetic field data, on the acquired data (Step S2).

Then, the IED probability map calculation unit 502 calculates a probability map of the characteristic waveform (IED) (Step S3).

As for calculation of the IED probability map, it may be possible to adopt a conventional method using a detection algorithm for detecting a spike position (see A. Ossadtchi, S. Baillet, J. Mosher, D. Thyerlei, W. Sutherling, and R. Leahy, “Automated interictal spike detection and source localization in magnetoencephalography using independent components analysis and spatio-temporal clustering”, Clinical Neurophysiology, vol. 115, no. 3, pp. 508-522, 2004), in addition to a method of application of a model that is calculated by using machine learning. When the machine learning is to be used, training is performed by adopting a time point of an IED that is manually detected by a doctor as a correct answer, and therefore, as compared to the conventional method of detecting a spike position, it is possible to obtain a result that is similar to a result obtained by analysis of the doctor. In the conventional method, development is performed so as to detect an onset site of the IED or a peak of the IED; however, in actual analysis, a doctor performs analysis while adjusting a time at which dipole estimation is performed in a range from the onset site to the peak by viewing a waveform, diagnosis and opinion, or the like.

Here, a case will be described in which the IED probability map calculation unit 502 uses machine learning for generation of the IED probability map.

FIG. 6 is a flowchart illustrating the flow of a training process in a case where the IED probability map is generated by using machine learning. As illustrated in FIG. 6, the IED probability map calculation unit 502 acquires pre-processed magnetoencephalography data that is subjected to the pre-processing by the pre-processing unit 501 (Step S11), performs learning on the pre-processed magnetoencephalography data in order to output the IED probability map (Step S12). Meanwhile, as correct answer data used for training, information on a sampling time point that is recorded when dipole estimation is performed and a sensor that is used for the dipole estimation is used.

Specifically, it is possible to apply a network that is frequently used in a task called Semantic Segmentation as represented by U-Net described in O. Ronneberger P. Fischer and T. Brox “U-net: Convolutional networks for biomedical image segmentation,” Proc. Int. Conf. Medical Image Comput. Comput.-Assisted Intervention, pp. 234-241 2015. Semantic Segmentation indicates a task for performing labeling on each of elements in all of arrays to be input, and, in a medical image, Semantic Segmentation is used when a region of a brain tumor or a cancer is automatically estimated. In this example, training is performed such that input magnetoencephalography data corresponds to mask data, which is generated from a sensor that is selected when the doctor performs dipole estimation and an estimation time, while adopting the mask data as a correct answer. At this time, to improve generalization ability, it may be possible to adopt a data augmentation method, such as rearrangement of sensors in a random order or setting of a value in a certain range of a specific sensor to zero.

Furthermore, it is possible to similarly adopt a network that is used in a different task, such as Object Detection or Instance Segmentation, other than Semantic Segmentation. Object Detection is a method of estimating, by a rectangle, a position at which a target object is present in input arrays, and when this method is to be used, it is impossible to calculate the IED probability map, but it is possible to directly acquire a time at which dipole estimation is performed from the detected rectangle (for example, a midpoint of the rectangle) and a sensor. Instance Segmentation is a task in which Semantic Segmentation and Object Detection are mixed, and it is possible to calculate a region of a detected Object, in addition to classification and the number of the detected Object. Instance Segmentation is able to calculate the IED probability map, and therefore, can be applied as it is.

Subsequently, after the training at Step S2 is completed, the IED probability map calculation unit 502 generates a trained model (Step S13). The generated trained model is used for inference of machine learning, that is, when an IED is to be detected from unknown data.

The IED probability map that is calculated by the IED probability map calculation unit 502 will be described below.

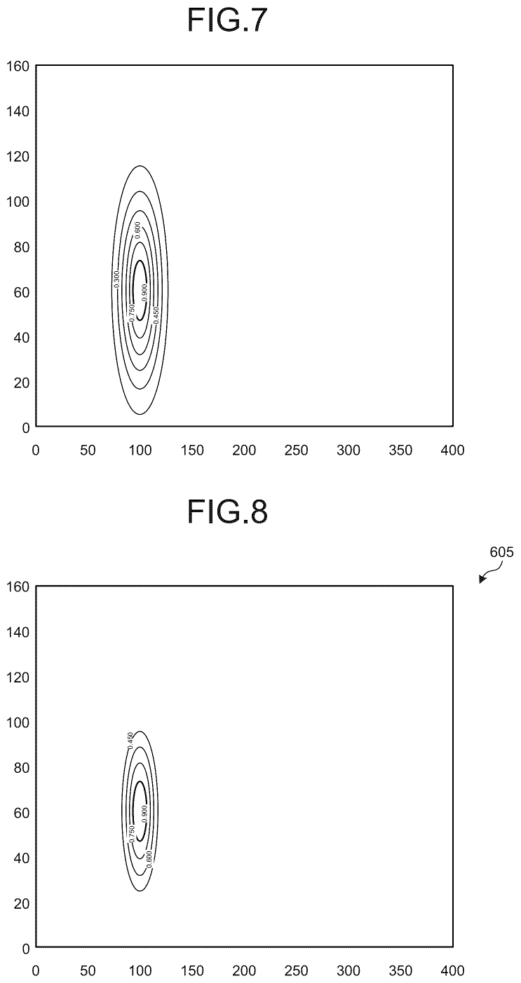

FIG. 7 is a diagram illustrating an example of an IED probability map. In FIG. 7, a horizontal axis represents an index of a time and a vertical axis represents an index of a sensor. In the example illustrated in FIG. 7, it is estimated that IEDs appear when an index of the time is 100 and an index of the sensor is about 40 to 80. Meanwhile, in FIG. 7, the probabilities of IEDs are represented by gradation of black and white, but it may be possible to use colors such that colors are changed in accordance with the probabilities of IEDs.

Referring back to FIG. 5, the threshold processing unit 503 performs a threshold process on the IED probability map that is acquired by the IED probability map calculation unit 502 (Step S4). Specifically, the threshold processing unit 503 narrows down, by using a threshold, a region of a time and sensors in which the probabilities of IEDs are high from the IED probability map that is obtained by the IED probability map calculation unit 502.

Meanwhile, in the threshold processing unit 503, it is possible to select only waveforms that are more likely to be IEDs with an increase in the threshold; however, the number of IEDs used for dipole estimation is reduced. In contrast, in the threshold processing unit 503, if the threshold is reduced, it is possible to detect an increased number of IEDs although erroneous detection increases. Further, magnetoencephalography data is largely affected by individual differences, and therefore, if the threshold is not reduced, there may be a case in which IED is less likely to be detected.

Therefore, the threshold processing unit 503 may be configured to interactively control the number of IEDs to be detected in cooperation with a User Interface (UI).

The threshold processing unit 503 basically uses a preset value, such as “0.8”, as a threshold.

FIG. 8 is a diagram illustrating an example of the IED probability map that is subjected to the threshold process. In the example illustrated in FIG. 8, the threshold processing unit 503 uses “0.5” as a threshold. In an IED probability map 605 that is a two-dimensional map illustrated in FIG. 8, a portion in which an IED is present is extracted with the probability of 50% or more.

Referring back to FIG. 5, the post-processing unit 504 performs post-processing for extracting a sampling time point and a sensor that are used for dipole estimation from the IED probability map 605 that is subjected to the threshold process by the threshold processing unit 503 (Step S5).

Specifically, if the threshold processing unit 503 is applied, a region of a time and sensors, which includes values equal to or larger than a predetermined probability, is extracted. To clarify a time point of peak detection or IED detection, it may be possible to apply a certain filter, such as a Gaussian filter, on the extracted region of sensors before peak detection. By detecting a peak after applying a certain filter, such as a Gaussian filter, to the extracted region of sensors, it is possible to determine the time point of the IED. When detecting a peak, it may be possible to adopt a one-dimensional probability map in which the probability map including the extracted region of sensors is subjected to additive average in a sensor direction. When additive average is not performed, it may be possible to detect a peak for each of the sensors and determine the time of the IED by obtaining an average of the times.

The threshold processing unit 503 extracts sensors that have values equal to or larger than a predetermined probability at the detected peak time points, as sensors that are used for dipole estimation. In this case, it is known that, if the number of sensors used for dipole estimation is reduced, stability of a dipole estimated solution is reduced. Therefore, in this example, when the number of the selected sensors is smaller than a predetermined number, it may be possible not to perform dipole estimation at this time point of the IED.

Furthermore, as another sensor extraction method, it may be possible to define a group of sensors in advance and augment an extracted sensor to all of the sensors in a group to which the extracted sensor belongs, so that it is possible to improve stability of a dipole estimated solution. Here, it is assumed that the group is basically formed in accordance with an anatomical criterion, such as near a temporal lobe or near a frontal lobe; however, it may be possible to simply adopt a neighboring region including an arbitrary number of sensors as a single group.

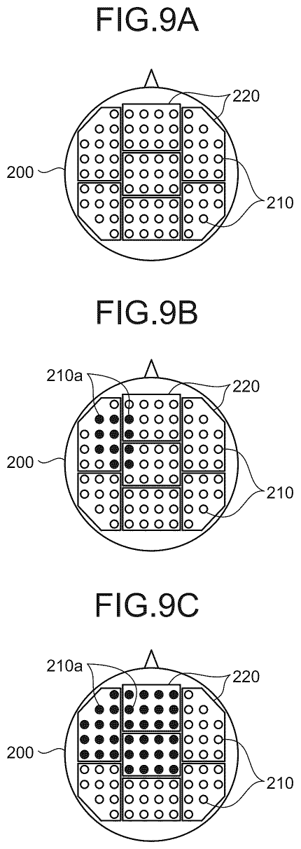

FIGS. 9A to 9C are schematic diagrams illustrating an example of a sensor augmentation method. FIG. 9A illustrates a head portion 200 of a subject, magnetoencephalography sensors 210 that are arranged so as to cover the head portion 200, and groups 220 of the sensors 210 that are set in advance. In FIG. 9B, sensors 210a represented by black circles are sensors that are extracted by the threshold process. It may be possible to estimate a dipole by using only the sensors 210a that are extracted by the threshold process; however, as described above, it may be possible to augment sensors in order to improve stability of a dipole estimated solution. FIG. 9C illustrates a result of augmentation of the number of sensors. Specifically, as illustrated in FIG. 9C, the sensors 210a that are extracted by the threshold process are augmented to all of sensors 210 in the groups 220 to which the sensors 210a belong. With this configuration, it is possible to improve stability of the dipole estimated solution.

Furthermore, if the IED probability map includes a plurality of peaks (a case in which two or more lesion sites are present, for example) the post-processing unit 504 may perform post-processing (to be described later) for separating the map.

As another example of setting of the neighboring region, it may be possible to construct a convex hull from the coordinates of the selected sensors, expand a space by performing Morphological transformation on the convex hull by a predetermined threshold, and adopt sensors included in the expanded space as a single group.

Furthermore, it may be possible to sequentially acquire sensors from the nearest neighbor point with reference to a midpoint of spatial coordinates of a sensor (source) with a maximum value of a magnetic field of the selected sensor data and a sensor (sink) with a minimum value.

Moreover, it may be possible to reconstruct a spatial isomagnetic contour map by a predetermined magnetic field threshold from magnetic field information that is acquired from the sensor data, and select a sensor that is near the magnetic field information on the already-selected sensor and that belongs to the same isomagnetic line from the magnetic field information on the selected sensor.

Furthermore, it may be possible to form a group by combining a determination means using a spatial distribution of the magnetic field of the neighboring region and an anatomical criterion, and form a sensor group from the spatial and anatomical viewpoint.

The peak detection unit 505 detects a peak by using a sampling point that is obtained by a prior process (Step S6). For example, the peak detection unit 505 has a function to detect a waveform with a specific pattern hidden in noise to detect peak candidates by pattern matching using machine learning or the like. The peak detection unit 505 has a function to determine a peak by using a score that is quantified by a predetermined procedure conditions when detecting the peak candidates. In the present embodiment, the “score” is used in which a concept, such as intensity of pattern matching or a precision ratio of two waveforms, is quantified.

Lastly, the estimation time detection unit 506 detects a current source estimation time by using the peak time that is obtained by the peak detection unit 505 (Step S7).

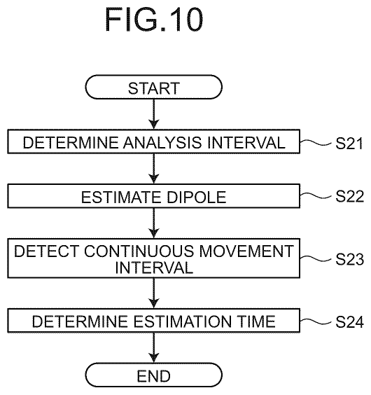

A flow of detection of an estimation time by the estimation time detection unit 506 will be described below. FIG. 10 is a flowchart illustrating the flow of an estimation time detection process.

As illustrated in FIG. 10, the flow of the estimation time detection process performed by the estimation time detection unit 506 includes an analysis interval determination step S21, a dipole estimation step S22, a continuous movement interval detection step S23, and an estimation time determination step S24.

First, at the analysis interval determination step S21, the estimation time detection unit 506 sets a time with a fixed width as an interval in which a characteristic waveform is obtained by adopting the peak time of the waveform detected by the peak detection unit 505 as a terminal point, and adopts the set time as an analysis interval. It is desirable to set a duration of the analysis interval by taking into account statistical information on a duration from the onset site to the peak of a spike waveform (Nowak, Rafal, et al. “Toward a Definition of Meg Spike: Parametric Description of Spikes Recorded Simultaneously by Meg and Depth Electrodes”. Seizure, vol. 18, no. 9, pp. 652-655, 2009). In addition, it may be possible to allow a doctor or an engineer to set a desired duration.

Subsequently, at the dipole estimation step (current source estimation step) S22, the estimation time detection unit 506 performs dipole estimation at all of sampling points on a signal in the interval that is determined at the analysis interval determination step S21 (see M. Scherg, “Fundamentals of dipole source potential analysis”, in Auditory Evoked Magnetic Fields and Potentials, M. Hoke, F. Grandori, and G. L. Romani, Eds. Basel, Switzerland: Karger, 1989, vol. 6). In this example, it is possible to adopt, other than the dipole estimation, a current source analysis method using a spatial filtering method, such as the minimum norm method (see K. Sekihara, M. Sahani, and S. S. Nagarajan, “Localization bias and spatial resolution of adaptive and non-adaptive spatial filters for MEG source reconstruction”, NeuroImage, vol. 25, no. 4, pp. 1056-1067, 2005) or the LCMV Beamformer method (see B. V. Veen, W. V. Drongelen, M. Yuchtman, and A. Suzuki, “Localization of brain electrical activity via linearly constrained minimum variance spatial filtering”, IEEE Transactions on Biomedical Engineering, vol. 44, no. 9, pp. 867-880, 1997). The spatial filtering method is a method in which approximately tens of thousands of dipoles are arranged in a brain in advance, and it is possible to obtain a temporal change of an electric current in each of the dipoles.

Subsequently, at the continuous movement interval detection step S23, the estimation time detection unit 506 extracts an interval in which a movement amount of a dipole is small (continuously movement interval) with reference to the peak time in the analysis interval by using the coordinates of the dipole (current source) that is estimated at the dipole estimation step S22.

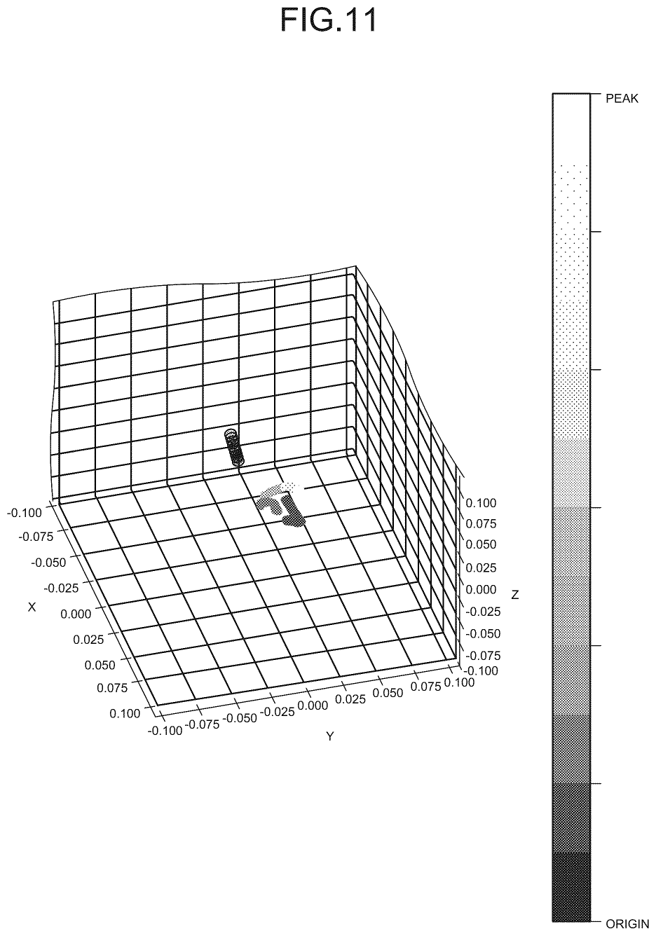

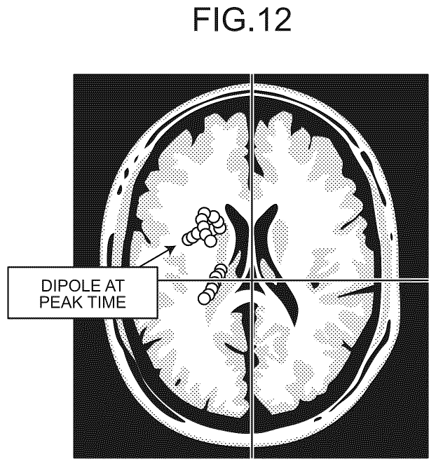

FIG. 11 is a diagram illustrating a trajectory of a dipole in the analysis interval, and FIG. 12 is a diagram illustrating an example of display in which the trajectory of the dipole in the analysis interval is superimposed on a brain slice image. For example, FIG. 11 illustrates an example of coordinates of a dipole for which dipole estimation is performed at all of sampling points in the analysis interval. Further, FIG. 12 illustrates an example in which the dipole in FIG. 11 are superimposed on a brain slice image.

In FIG. 11 and FIG. 12, when the way of movement of the dipole in a time direction from the peak to the onset site is payed attention to, a movement amount per sample is small in a certain interval from the peak time and the dipole moves in a continuous manner. Thereafter, it is found that the position of the dipole is largely changed and the dipole moves in a continuous manner. In this example, the estimation time detection unit 506 adopts an interval from the peak time to a point immediately before the position of the dipole is largely changed, as a

Examples of a method of extracting a continuous movement interval include the following methods.
(1) Method of extracting an interval in which a distance of a dipole from a previous sampling point does not exceed a threshold
(2) Method of extracting an interval in which a movement distance of a dipole in a certain constant sampling width does not exceed a threshold
(3) Method of extracting an interval in which a sum of cumulative movement does not exceed a threshold when the peak time is adopted as a start point

Meanwhile, it may be possible that a sampling frequency varies depending on a recording condition, and therefore, it may be possible to normalize a sampling interval in the methods as described above and use a movement distance of the dipole or the like for each certain fixed time.

Lastly, at the estimation time determination step S24, the estimation time detection unit 506 determines a final single dipole estimation time (current source estimation time) from the continuous movement interval.

Examples of a method of determining the dipole estimation time from the continuous interval include the following methods.
(1) Method of adopting a beginning time of the continuous movement interval as the estimation time
(2) Method of adopting a time a fixed time later than the beginning time of the continuous estimation interval as the estimation time
(3) Method of adopting a time a fixed percent of time later the beginning time of the continuous estimation interval (for example, one-third of the time of the continuous estimation interval) as the estimation time
(4) Method of adopting a time a fixed time earlier than a terminal end (peak time) of the continuous estimation interval as the estimation time
(5) Method of adopting a time a fixed percent of time earlier than the beginning time of the continuous estimation interval (for example, two-third of the time of the continuous estimation interval) as the estimation time

Through the flow as described above, the estimation time detection unit 506 searches for an interval in which an activity of a characteristic waveform is captured and selects a start time in the interval, so that it is possible to automatically detect a time corresponding to the onset site of the activity with respect to the activity of the characteristic waveform.

As described above, according to the present embodiment, an analysis interval that includes characteristic waveform information is detected from waveform data that is acquired by a sensor, and an interval in which a current source (dipole) moves in a continuous manner in the analysis interval is retrieved. With this configuration, a time for estimation of the current source is determined while taking into account the onset site of the characteristic waveform information (IED), so that it is possible to determine the time of the characteristic waveform information (IED) in more accordance with an analysis method adopted by a doctor.

### Second Embodiment

A second embodiment will be described below.

A current source estimation time determination method according to the second embodiment will be described below mainly in terms of differences from the current source estimation time determination method according to the first embodiment. In the explanation of the second embodiment below, explanation of the same components as those of the first embodiment will be omitted, and differences from the first embodiment will be described.

In the present embodiment, at the estimation time determination step S24 performed by the estimation time detection unit 506 of the first embodiment, selection is performed among the estimated dipoles based on an evaluation index.

More specifically, the estimation time detection unit 506 of the present embodiment sets a threshold for an evaluation index for dipole estimation, where the evaluation index includes at least one of Goodness of Fit (GoF) that represents a degree of goodness of approximation of the dipole estimation, correlation, Confidence Volume, and a current moment (intensity) of the estimated dipole. The estimation time detection unit 506 of the present embodiment further extracts times at each of which the evaluation index for the dipole falls within a threshold in the continuous movement interval that is acquired at the previous step, and selects a final estimation time from among the extracted times.

To evaluate validity of a dipole estimation result, the estimation time detection unit 506 may use a causal analysis result (Causality) of sequential data or a network (Vaudano, Anna Elisabetta, et al. “Causality within the Epileptic Network: An EEG-fMRI Study Validated by Intracranial EEG”. Frontiers in Neurology, vol. 4, 2013), frequency coupling for evaluating synchronous activities in different frequency bands (Edakawa, Kohtaroh, et al. “Detection of Epileptic Seizures Using Phase-Amplitude Coupling in Intracranial Electroencephalography”. Scientific Reports, vol. 6, no. 1, 2016), or the like, in addition to the evaluation index for the dipole estimation.

In general, it is known that the S/N ratio is low at the onset site, and reliability of the dipole estimation result may be reduced. According to the estimation time detection unit 506 of the present embodiment, by providing a threshold for the evaluation index for the dipole estimation, it is possible to take into account the reliability of the dipole estimation.

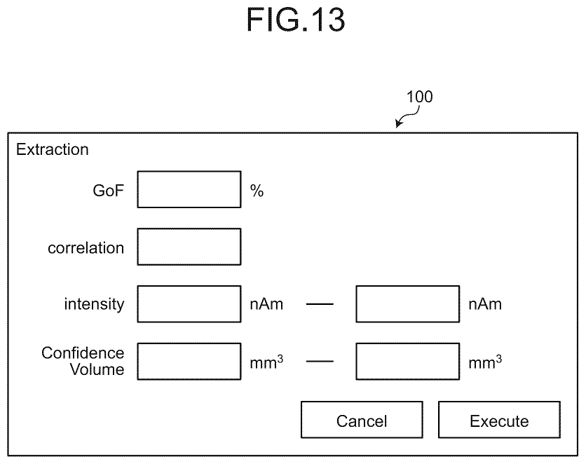

FIG. 13 is a diagram illustrating an example of a threshold setting input user interface (UI) according to the second embodiment. In a Graphical User Interface (GUI) screen 100 illustrated in FIG. 13, it is possible to set a threshold for each of GoF, correlation, a current moment (intensity) of the estimated dipole (intensity), and Confidence Volume as the threshold for the evaluation index for the dipole estimation. In this manner, by cooperation with the GUI screen 100 as illustrated in FIG. 13, it is possible to allow a doctor or an engineer to input an arbitrary value as the threshold for the evaluation index for the dipole estimation, and it is possible to provide a result in accordance with the input.

### Third Embodiment

A third embodiment will be described below.

A current source estimation time determination method according to the third embodiment will be described below mainly in terms of differences from the current source estimation time determination methods according to the first embodiment and the second embodiment. In the explanation of the third embodiment below, explanation of the same components as those of the first embodiment and the second embodiment will be omitted, and differences from the first embodiment or the second embodiment will be described.

In the present embodiment, at the continuous movement interval detection step S23 performed by the estimation time detection unit 506 of the first embodiment or the second embodiment, if a movement amount of the estimated dipole meets a predetermined condition (if the movement amount of the estimated dipole is small), the time at a terminal end of the analysis interval detected by the peak detection unit 505 is adopted for the estimation time, to detect the continuous movement interval.

As a specific method for determination on whether the movement amount of the dipole is small, the following methods may be adopted.
(1) Method for determination based on whether a cumulative movement of the dipole coordinates in the analysis interval falls in a range that does not exceed a threshold
(2) Method for determination based on whether a maximum movement distance with reference to coordinates of the dipole at a reference time in the analysis interval falls in a range that does not exceed a threshold
(3) Method for determination based on whether the dipole in the analysis interval is present in the same gyrus

According to the present embodiment, at the continuous movement interval detection step S23 performed by the estimation time detection unit 506, reliability of the dipole estimation result is increased when the dipole estimation is performed at a certain time at which the SN ratio is better; therefore, if a difference between an estimation position at the onset site and an estimation position at the peak is small, it is possible to obtain a result with higher reliability by performing the dipole estimation at the peak time.

Meanwhile, in each of the embodiments as described above, when at least any of the functional units in the biological signal measurement system 1 is implemented by execution of a program, the program is provided by being incorporated in a ROM or the like. Further, the program that is executed by the biological signal measurement system 1 according to the embodiment as described above may be provided by being recorded in a computer-readable recording medium, such as a CD-ROM, a flexible disk (FD), a Compact Disc Recordable (CD-R), or a Digital Versatile Disk (DVD) in a computer-installable or a computer-executable file format.

Furthermore, the program that is executed by the biological signal measurement system 1 according to each of the embodiments as described above may be stored in a computer that is connected to a network, such as the Internet, and may be provided by download via the network.

Moreover, the program that is executed by the biological signal measurement system 1 according to each of the embodiments as described above may be provided or distributed via a network, such as the Internet. Furthermore, the program that is executed by the biological signal measurement system 1 according to each of the embodiments as described above has a module structure including at least any of the functional units as described above, and as actual hardware, each of the functional units as described above is loaded and generated on a main storage device by causing a CPU to read and executes the program from the ROM or the like.

According to one aspect of the present invention, it is possible to determine a time of characteristic waveform information (IED) in more accordance with an analysis method adopted by a doctor.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A current source estimation time determination method comprising:
determining an analysis interval in which a characteristic waveform is obtained with respect to one or more pieces of waveform data acquired by a sensor;
estimating a current source with respect to a signal in the analysis interval determined at the determining;
detecting a continuous movement interval in which the current source continuously moves in the analysis interval, using coordinates of the current source estimated at the estimating; and
determining a single current source estimation time from the continuous movement interval detected at the detecting.

2. The current source estimation time determination method according to claim 1, wherein the determining includes setting a duration as an interval in which characteristic waveform information is obtained, and adopting the set duration as the analysis interval.

3. The current source estimation time determination method according to claim 1, wherein the determining includes determining the current source estimation time while taking into account an evaluation index for the current source.

4. The current source estimation time determination method according to claim 3, wherein the determining includes, as the evaluation index for the current source, at least one of Goodness of Fit (GoF), correlation, Confidence Volume, and a current moment (intensity) of the current source.

5. The current source estimation time determination method according to claim 3, wherein the determining includes using at least one of a causal analysis result (Causality) and frequency coupling, in addition to the evaluation index for the current source.

6. The current source estimation time determination method according to claim 1, wherein the detecting includes adopting time at a terminal end of the analysis interval if a movement amount of the current source meets a predetermined condition, to detect the continuous movement interval.

7. The current source estimation time determination method according to claim 6, wherein the detecting includes using, as the predetermined condition, at least one of:
a condition that a cumulative movement of the coordinates of the current source in the analysis interval does not exceed a threshold;
a condition that a maximum movement distance with reference to the coordinates of the current source at a reference time in the analysis interval does not exceed a threshold; and
a condition that the current source in the analysis interval is present in a same gyrus.

8. An information processing apparatus comprising
an estimation time detection unit configured to detect an estimation time of a current source, wherein
the estimation time detection unit is configured to:
determine an analysis interval in which a characteristic waveform is obtained with respect to one or more pieces of waveform data acquired by a sensor;
estimate a current source with respect to a signal in the determined analysis interval;
detect a continuous movement interval in which the current source continuously moves in the analysis interval, using coordinates of the estimated current source; and
determine a single current source estimation time from the detected continuous movement interval.

9. A non-transitory computer readable medium including programmed instructions that cause a computer to execute:
determining an analysis interval in which a characteristic waveform is obtained with respect to one or more pieces of waveform data acquired by a sensor;
estimating a current source with respect to a signal in the analysis interval determined at the determining;
detecting a continuous movement interval in which the current source continuously moves in the analysis interval, using coordinates of the current source that is estimated at the estimating; and
determining a single current source estimation time from the continuous movement interval detected at the detecting.
